(19) United States
(12) Patent Application Publication
(10) Pub. No.: US 2022/0310227 A1
(43) Pub. Date: Sep. 29, 2022

(54) Rehabilitation Support System, Rehabilitation Support Method, and Rehabilitation Support Program

(71) Applicant: Nippon Telegraph and Telephone Corporation, Tokyo (JP)

(72) Inventors: Takayuki Ogasawara, Tokyo (JP); Shin Toyota, Tokyo (JP); Masahiko Mukaino, Toyoake-shi (JP); Eiichi Saitho, Aichi (JP)

(21) Appl. No.: 17/635,758

(22) PCT Filed: Aug. 6, 2020
(86) PCT No.: PCT/JP2020/030224

§ 371 Date: Feb. 16, 2022

(30) Foreign Application Priority Data
Aug. 20, 2019 (JP) 2019-150202

(51) Int. Cl. G16H 20/30; G16H 40/63

(52) U.S. Cl. CPC G16H 20/30; G16H 40/63

(57) ABSTRACT

A rehabilitation support technique with which a user can be more motivated to work on his or her rehabilitation is provided. A rehabilitation support system includes: a sensor data acquirer configured to acquire biometric information on a user measured by a sensor; an estimator configured to estimate a state of the user based on the biometric information acquired; a storage device configured to store a mode of a spatiotemporally changing item; a selector configured to select the mode of the spatiotemporally changing item in accordance the state of the user estimated by the estimator, from modes of the spatiotemporally changing item stored in the storage device; and a presenter configured to present the mode of the spatiotemporally changing item selected by the selector.

### CROSS-REFERENCE TO RELATED APPLICATIONS

This application is a national phase entry of PCT Application No. PCT/JP2020/030224, filed on Aug. 6, 2020, which application claims priority to Japan Patent Application No. 2019-150202, filed on Aug. 20, 2019, which applications are hereby incorporated herein by reference.

### TECHNICAL FIELD

The present invention relates to a rehabilitation support system, a rehabilitation support method, and a rehabilitation support program.

### BACKGROUND

With a proper rehabilitation process, patients, elderly people, and the like who need rehabilitation can rehabilitate their physical functions, and achieve their targets in terms of psychological and social aspects regarding their quality of life. Patients requiring rehabilitation may need to aggressively perform rehabilitation over their entire waking hours, to enable them to recover from diseases or the like for example.

Biometric information such as heart rate and amount of activity measured by a sensor, such as a wearable terminal, has been utilized in the fields of sports and medicine (see, for example, PTL1 and NPL1). For example, PTL1 discloses a technique for analyzing a patient's state of activity more accurately by examining on their lifestyle, based on acceleration measured by a sensor attached to the user.

With the related art, the state of physical activities of the user such as a patient performing rehabilitation (hereinafter, simply referred to as “rehab”) can be recognized and such information can be presented. However, dynamic information has not been presented to the user when he or she works on the rehabilitation. Thus, with such a technique, it may be difficult to motivate the user who needs rehabilitation toward the rehabilitation over the entirety of his or her waking hours.

### CITATION LIST

### Patent Literature

PTL1: WO 2018/001740

### Non Patent Literature

NPL1: Kasai, Ogasawara, Nakajima, Tsukada, “Development and Application of Functional Material “hitoe” Enabling Measurement of Biometric Information When Worn”, IEICE Communication Society Magazine #41 (June 2017), (Vol. 11, No. 1)

### SUMMARY

### Technical Problem

The present invention has been made to solve the problem described above, and an object of the present invention is to provide a rehabilitation support technique with which a user can be more motivated to work on his or her rehabilitation.

### Means for Solving the Problem

A rehabilitation support system according to an embodiment of the present invention for solving the above problem includes: a sensor data acquisition unit configured to acquire biometric information on a user measured by a plurality of sensors; an estimation unit configured to estimate a state of the user based on the biometric information acquired; a storage unit configured to store a mode of a spatiotemporally changing item; a selection unit configured to select the mode of the item stored in the storage unit, in accordance the state of the user estimated by the estimation unit, from modes of the item stored in the storage unit; and a presentation unit configured to present the mode of the item selected by the selection unit.

In the rehabilitation support system according to an embodiment of the present invention, the presentation unit may include a display device configured to display an image representing the mode of the item in accordance with the state of the user.

In the rehabilitation support system according to an embodiment of the present invention, the sensor data acquisition unit may acquire biometric information on the user from each of a plurality of sensors, the estimation unit may estimate values of a plurality of parameters indicating the state of the user, based on the biometric information acquired from each of the plurality of sensors, and the selection unit may select the mode of the item in accordance with the estimated values of the plurality of parameters.

In the rehabilitation support system according to an embodiment of the present invention, the selection unit may select the mode of the item with weighting the estimated values of the plurality of parameters indicating the state.

In the rehabilitation support system according to an embodiment of the present invention, the plurality of parameters indicating the state may include an activity state, a magnitude of body movement, and a change in heart rate of the user.

The rehabilitation support system according to an embodiment of the present invention may further include a detection unit configured to detect whether the user is performing a rehabilitation, and the selection unit may select, when the detection unit detects that the user is performing the rehabilitation, the mode of the item in accordance with the state of the user estimated by the estimation unit, and select, when the detection unit detects that the user is not performing the rehabilitation, the mode of the item in accordance with a length of a period during which the user is not performing the rehabilitation.

A rehabilitation support method according to an embodiment of the present invention for solving the problem described above includes: a first step of acquiring biometric information on a user measured by a plurality of sensors; a second step of estimating a state of the user based on the biometric information acquired in the first step; a third step of selecting a mode of an item, in accordance with the state of the user estimated in the second step, from modes of the spatiotemporally changing item stored in a storage unit; and a fourth step of presenting the mode of the item selected in the third step.

A rehabilitation support program according to an embodiment of the present invention for solving the problem described above causes a computer to execute: a first step of acquiring biometric information on a user measured by a plurality of sensors; a second step of estimating a state of the user based on the biometric information acquired in the first step; a third step of selecting a mode of an item, in accordance with the state of the user estimated in the second step, from modes of the spatiotemporally changing item stored in a storage unit; and a fourth step of presenting the mode of the item selected in the third step.

### Effects of Embodiments of the Invention

With embodiments of the present invention, based on biometric information on a user, a state of the user is estimated, and a mode of a spatiotemporally changing item is selected and presented based on the state of the user estimated, so that the user can be more motivated to work on his or her rehabilitation.

### BRIEF DESCRIPTION OF THE DRAWINGS

FIG. 1 is a block diagram illustrating a functional configuration of a rehabilitation support system according to a first embodiment of the present invention.

FIG. 2 is a block diagram illustrating a configuration of a data analysis unit according to the first embodiment.

FIG. 3 is a block diagram illustrating an example of a computer configuration that implements the rehabilitation support system according to the first embodiment.

FIG. 4 is a flowchart illustrating an operation of the rehabilitation support system according to the first embodiment.

FIG. 5 is a diagram illustrating an outline of an example of a configuration of the rehabilitation support system according to the first embodiment.

FIG. 6 is diagram illustrating a display example of rehabilitation support information according to the first embodiment.

FIG. 7 is a diagram illustrating an example of a mode of an image according to the first embodiment.

FIG. 8 is a block diagram illustrating an example of a configuration of the rehabilitation support system according to the first embodiment.

FIG. 9 is a sequence diagram illustrating an operation of the rehabilitation support system according to the first embodiment.

FIG. 10 is a block diagram illustrating a configuration of a data analysis unit according to a second embodiment.

FIG. 11 is a flowchart illustrating an operation of the rehabilitation support system according to the second embodiment.

FIG. 12 is diagram illustrating a display example of rehabilitation support information according to the second embodiment.

FIG. 13 is a block diagram illustrating a configuration of the rehabilitation support system according to a third embodiment.

FIG. 14 is a block diagram illustrating a configuration of a sensor data acquisition unit according to the third embodiment.

FIG. 15 is a flowchart illustrating an operation of the rehabilitation support system according to the third embodiment.

FIG. 16 is a block diagram illustrating a configuration of a data analysis unit according to a fourth embodiment.

FIG. 17 is a flowchart illustrating an operation of the rehabilitation support system according to the fourth embodiment.

FIG. 18 is a flowchart illustrating an operation of the rehabilitation support system according to the fifth embodiment.

### DETAILED DESCRIPTION OF ILLUSTRATIVE EMBODIMENTS

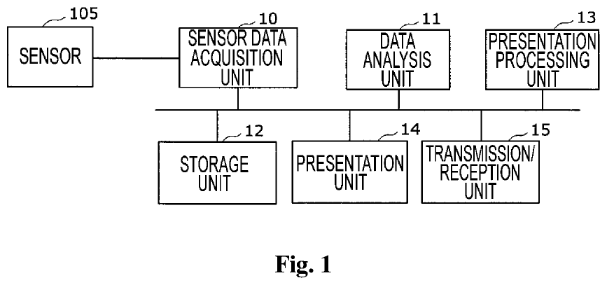

Preferred embodiments of the present invention will be described below in detail with reference to FIGS. 1 to 18.

### First Embodiment

First, an outline of a configuration of a rehabilitation support system according to a first embodiment of the present invention will be described. FIG. 1 is a block diagram illustrating a functional configuration of the rehabilitation support system. The rehabilitation support system acquires biometric information on a user measured by a sensor 105 to estimate a state of a user involved in rehabilitation. Based on the state of the user thus estimated, the rehabilitation support system selects the mode of a spatiotemporally changing item in accordance with the progress of the rehabilitation, and presents the mode of the item as rehabilitation support information.

### Functional Block of Rehabilitation Support System

The rehabilitation support system includes a sensor data acquisition unit 10 that acquires data from the sensor 105, a data analysis unit 11, a storage unit 12, a presentation processing unit 13, a presentation unit 14, and a transmission/reception unit 15.

The sensor data acquisition unit 10 acquires the biometric information on the user measured by the sensor 105. More specifically, when an acceleration sensor, as the sensor 105, is attached to the user, the sensor data acquisition unit 10 converts an analog acceleration signal measured by the acceleration sensor into a digital signal at a predetermined sampling rate. The biometric information measured by the sensor data acquisition unit 10 is stored in the storage unit 12, which is described later, in association with a measurement time point.

The sensor data acquisition unit 10 may acquire, as the biometric information on the user, angular velocity, light, electromagnetic waves, temperature and humidity, pressure, position information, sound, concentration, voltage, resistance, and the like, in addition to acceleration. Furthermore, the sensor data acquisition unit 10 may acquire, as the biometric information on the user, cardiac electrical activity, myoelectric activity, blood pressure, intrabody gas exchanged by respiration, body temperature, pulse, and brainwave obtained from these physical quantities.

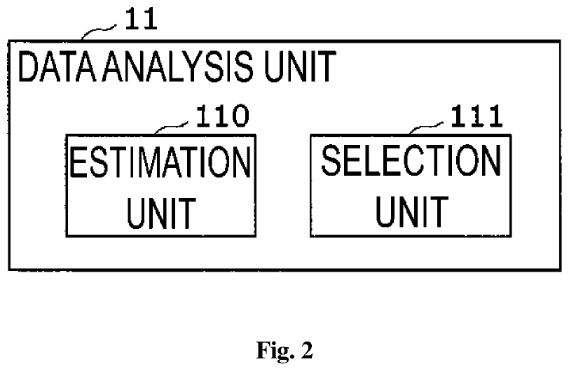

The data analysis unit 11 analyzes the biometric information on the user acquired by the sensor data acquisition unit 10 to estimate the state of the user involved in the rehabilitation, and selects the mode of the spatiotemporally changing item, in accordance with the estimated state of the user. As illustrated in FIG. 2, the data analysis unit 11 includes an estimation unit 110 and a selection unit 111.

The estimation unit 110 calculates the state of the user from the biometric information on the user acquired by the sensor data acquisition unit 10. The state of the user refers to the action, the posture, coordinates, speed, speech, breathing, walking, seating, driving, sleeping, body movement, stress, and the like involved in the rehabilitation performed by the user. Furthermore, the calculation may be performed to obtain other results which are information indicating quantity such as the magnitude, frequency, increase/decrease, duration, and accumulation of such states.

Specifically, the estimation unit 110 may estimate the state of the user using, for example, an out-of-bed state and a lying state estimated using the acceleration of the user described in PTL1. With the state of the user estimated by the estimation unit 110, the progress of the user's rehabilitation can be recognized.

The estimation unit 110 estimates the state of the user based on the biometric information on the user acquired over a period of time from the start of the measurement with the sensor 105 attached to the user to the current measurement time point. The result of estimating the state of the user by the estimation unit 110 is stored in the storage unit 12 together with time information.

The selection unit 111 selects the spatiotemporally changing mode of the item stored in the storage unit 12, in accordance with the state of the user estimated by the estimation unit no. More specifically, the selection unit 111 selects an image representing the mode of the spatiotemporally changing item, by using a history of the state within any period from the start of the measurement of the biometric information on the user performing the rehabilitation. For example, when the estimation unit no estimates that the user is in the out-of-bed state, a scene of a movie or the like is selected using a state history indicating a period during which the user is in the out-of-bed state.

The spatiotemporally changing item is information presented to the user as rehabilitation support information. Hereinafter, the mode of the spatiotemporally changing item and information including the same may be referred to simply as the rehabilitation support information.

When a user who is often in the lying state performs rehabilitation of getting out of bed, as the spatiotemporally changing item, for example, movie, sound, text, and combinations thereof can be used that represent the progress of the rehabilitation relative to a target value set in accordance with a frequency of the out-of-bed state or the out-of-bed time. Further, information presented in a form perceptible by the user such as vibration, heat, light, wind, or the like may be added to the movie or the like. Furthermore, a stereoscopic image such as a hologram may be used as the image.

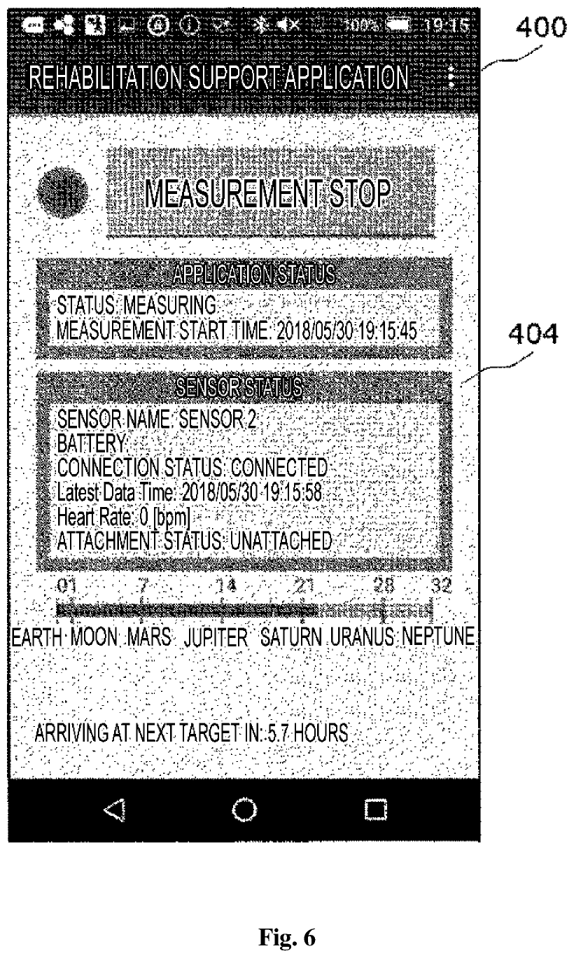

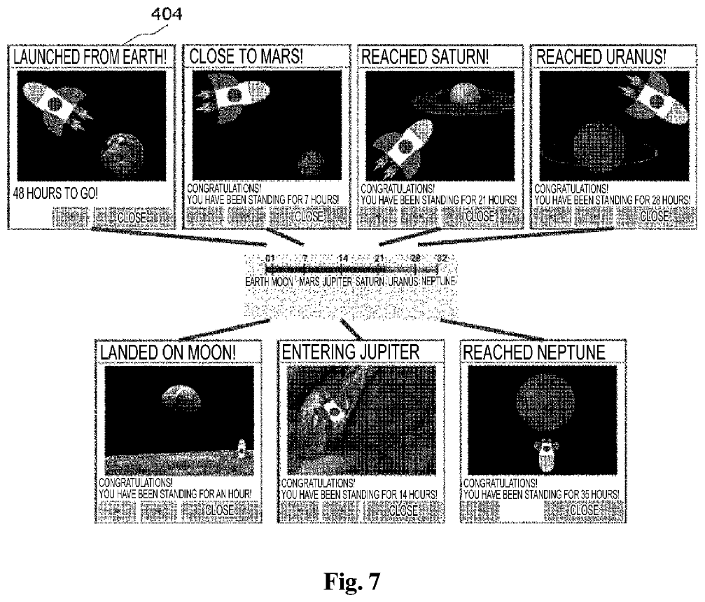

Specifically, as illustrated in FIGS. 6 and 7, a movie of a space craft traveling in space can be used as the spatiotemporally changing item presented as the rehabilitation support information. For example, when the user performs rehabilitation such as getting out of bed, the selection unit 111 selects image and text information indicating that the space craft has been launched from the earth, at the point when the measurement by the sensor 105 starts. Thereafter, the mode of the presented image is selected with the arrival point of the space craft switched, each time the out-of-bed time reaches a certain period of time, to Mars, Jupiter, Saturn, and Neptune which is the final destination farthest from the earth, in this order.

The rehabilitation support information illustrated in FIGS. 6 and 7 is an example, and the mode of the rehabilitation support information selected by the selection unit 111 is not limited to scenes of an animation of the space craft traveling. For example, the progress of the rehabilitation started by the user reaching a target set in advance may be represented by a movie or a still image such as an image indicating a process of building a building or a monument, an image indicating the growth of a plant or an animal, an image indicating actions of a character such as a person, and an animation indicating a musical performance, a competition held, solution of a puzzle, acquisition of honors and reward, as well as by text information, sound, and the like added to the image.

Information indicating the mode of the spatiotemporally changing item selected by the selection unit 111 is input to the presentation processing unit 13.

The storage unit 12 stores the mode of the spatiotemporally changing item. More specifically, the storage unit 12 can store in advance an image that changes in accordance with the progress of the rehabilitation described above. Information indicating the progress of the rehabilitation is associated with each image. For example, information indicating that the out-of-bed state has continued for a total time of an hour is stored in association with an image of the space craft reaching the moon.

The storage unit 12 stores time series data on biometric information on the user acquired by the sensor data acquisition unit 10. The storage unit 12 stores a history of the state of the user estimated by the estimation unit 110. The state of the user thus estimated is stored in the storage unit 12 together with the measurement time of the biometric information on which the state is based.

The presentation processing unit 13 generates the image presented by the presentation unit 14, based on the information indicating the mode of the spatiotemporally changing item selected by the selection unit 111. More specifically, the presentation processing unit 13 generates a movie presented as the rehabilitation support information, by using a still image of a format such as jpg, png, or bpm or a movie of a format such as gif, flash, or mpg as an image of a format set in advance. The presentation processing unit 13 can also generate rehabilitation support information such as sound or text presented in combination with the movie, as described above.

The presentation unit 14 causes a display device 109 described later to display the rehabilitation support information generated by the presentation processing unit 13. The presentation unit 14 switches the image displayed by the display device 109 based on a signal from the presentation processing unit 13.

The transmission/reception unit 15 receives sensor data indicating the biometric information on the user measured by the sensor 105. The transmission/reception unit 15 may convert information indicating the rehabilitation support information selected by the data analysis unit 11 according to a predetermined communication standard, and transmit the information to the presentation unit 14 connected via the communication network.

### Configuration of Computer for Rehabilitation Support System

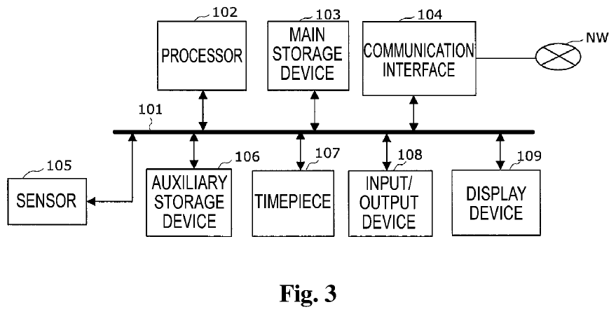

Next, an example of a computer configuration for achieving the rehabilitation support system having the functions described above will be described with reference to FIG. 3.

As illustrated in FIG. 3, the rehabilitation support system may be achieved by, for example, a computer including a processor 102, a main storage device 103, a communication interface 104, an auxiliary storage device 106, a timepiece 107, and an input/output device 108 connected to each other through a bus 101, and a program for controlling these hardware resources. The rehabilitation support system has, for example, the display device 109 provided therein and the sensor 105 provided outside the rehabilitation support system connected to each other via the bus 101.

The main storage device 103 stores in advance programs for the processor 102 to perform various controls and calculations. The processor 102 and the main storage device 103 achieve the functions of the rehabilitation support system including the data analysis unit 11 as illustrated in FIG. 1 and FIG. 2.

The communication interface 104 is an interface circuit for communicating with various external electronic devices via a communication network NW.

Examples of the communication interface 104 include an arithmetic interface and an antenna that comply with wireless data communication standards such as LTE, 3G, a wireless LAN, and Bluetooth (trade name). The transmission/reception unit 15 illustrated in FIG. 1 is achieved by the communication interface 104.

The sensor 105 includes, for example, a heart rate meter, an electrocardiograph, a blood pressure meter, a pulse rate meter, a respiration sensor, a thermometer, a brainwave sensor, and the like. More specifically, the sensor 105 is achieved by a three-axis acceleration sensor, a microwave sensor, a pressure sensor, a current meter, a voltmeter, a thermo-hygrometer, a concentration sensor, a photosensor, or a combination thereof.

The auxiliary storage device 106 is configured of a readable and writable storage medium, and a drive device for reading or writing various types of information such as programs or data from or to the storage medium. A hard disk or a semiconductor memory such as a flash memory can be used as a storage medium in the auxiliary storage device 106.

The auxiliary storage device 106 includes a storage region for storing the biometric information measured by the sensor 105 and a program storage region for storing a program for the rehabilitation support system to implement analysis processing on the biometric information. The storage unit 12 illustrated in FIG. 1 is achieved by the auxiliary storage device 106. Further, for example, a backup area for backing up the data, programs, and the like described above may be provided.

The timepiece 107 includes a built-in timepiece or the like of the computer and clocks a time. Alternatively, the timepiece 107 may acquire time information from a time server not illustrated in the drawing. The time information obtained by the timepiece 107 is recorded in association with the state of the user estimated. The time information obtained by the timepiece 107 is used for sampling of the biometric information or the like.

The input/output device 108 includes an I/O terminal that receives a signal from an external device such as the sensor 105 and the display device 109 and outputs a signal to an external device.

The display device 109 is implemented by a liquid crystal display or the like. The display device 109 achieves the presentation unit 14 illustrated in FIG. 1.

Rehabilitation Support Method

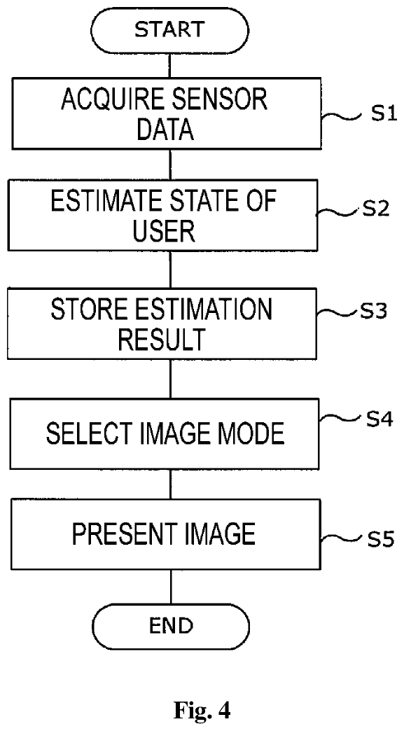

Next, the operation of the rehabilitation support system configured as described above will be described with reference to a flowchart of FIG. 4. First, the following processing is executed in a state where the sensor 105 is attached to the user for example.

The sensor data acquisition unit 10 acquires the biometric information on the user measured by the sensor 105 via the transmission/reception unit 15 (step S1). The acquired biometric information is accumulated in the storage unit 12. Note that the sensor data acquisition unit 10 can execute processing of remove the noise in the biometric information acquired, and converting the analog signal of the biometric information into a digital signal.

Next, the estimation unit no estimates the state of the user based on the biometric information on the user acquired by the sensor data acquisition unit 10 (step S2). For example, the estimation unit no estimates that the user is in the out-of-bed state from the data indicating the acceleration of the user acquired by the sensor data acquisition unit 10. The result of the estimation by the estimation unit no is stored in the storage unit 12 along with the time information (step S3).

Thereafter, the selection unit 111 selects a mode of the spatiotemporally changing item to be presented as the rehabilitation support information, by using a history within any period from the start of measurement of sensor data based on which the state of the user that performs the rehabilitation is estimated by the estimation unit no (step S4). For example, as illustrated in FIG. 6 and FIG. 7, a case is considered in which a user who is often in the lying state gets out of bed for rehabilitation. In this case, a movie of the space craft that has departed from the earth traveling in space to reach Neptune is used as the rehabilitation support information indicating the length of a period during which the user is in the out-of-bed state. The selection unit 111 selects, using a history of the state of the user stored in the storage unit 12, an image of a planet, which is a passing point of the space craft, corresponding to the time during which the user is in the out-of-bed state.

Next, the presentation unit 14 causes the display device 109 to display the mode of the spatiotemporally changing item as the rehabilitation support information, based on the mode of the spatiotemporally changing item selected by the selection unit 111 (step S5). More specifically, the presentation processing unit 13 generates an image, sound, and text corresponding to the mode of the spatiotemporally changing image selected by the selection unit iii. The presentation unit 14 outputs the image of the mode generated by the presentation processing unit 13 as the rehabilitation support information.

### Specific Configuration of Rehabilitation Support System

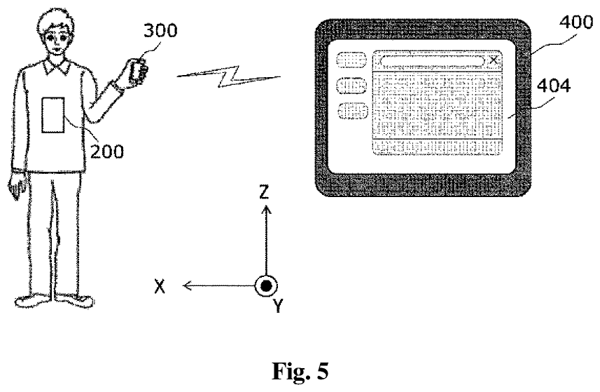

Next, an example of a specific configuration of the rehabilitation support system according to embodiments of the present invention will be described with reference to FIG. 5 to FIG. 8.

For example, as illustrated in FIG. 5, the rehabilitation support system includes a sensor terminal 200 attached to a user that performs the rehabilitation, a relay terminal 300, and an external terminal 400. All or any of the sensor terminal 200, the relay terminal 300, and the external terminal 400 includes the functions included in the rehabilitation support system such as the data analysis unit 11 illustrated in FIG. 1 and FIG. 2. The following description is given under an assumption that the relay terminal 300 includes the data analysis unit 11 illustrated in FIG. 1, and the rehabilitation support information is presented on the external terminal 400.

### Functional Block of Sensor Terminal

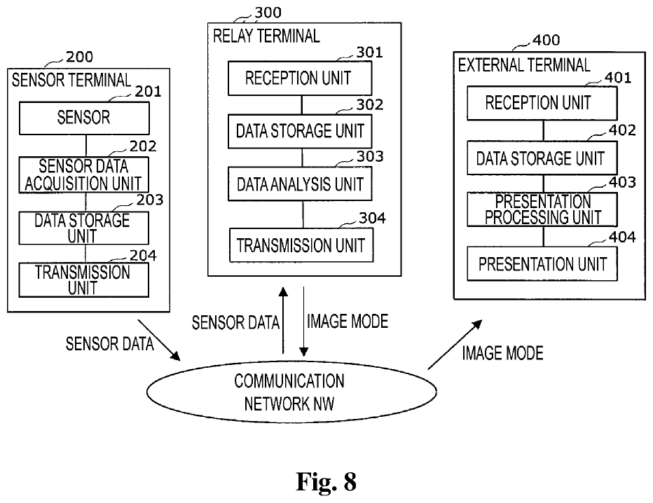

The sensor terminal 200 includes a sensor 201, a sensor data acquisition unit 202, a data storage unit 203, and a transmission unit 204 as illustrated in FIG. 8. For example, the sensor terminal 200, which is placed on the trunk of the body of the user 500, measures biometric information. The sensor terminal 200 transmits the measured biometric information on the user 500 to the relay terminal 300 through the communication network NW.

The sensor 201 is achieved by the three-axis acceleration sensor and the like for example. Regarding the three axes of the acceleration sensor included in the sensor 201, the X axis is provided in parallel with the right-left direction of the body, the Y axis is provided in parallel with the front-back direction of the body, and the Z axis is provided in parallel with the up-down direction of the body, for example, as illustrated in FIG. 5. The sensor 201 corresponds to the sensor 105 described in FIG. 1.

The sensor data acquisition unit 202 acquires the biometric information measured by the sensor 201. More specifically, the sensor data acquisition unit 202 performs noise removal and sampling processing on the biometric information acquired, and obtains time series data on the biometric information of the digital signal. The sensor data acquisition unit 202 corresponds to the sensor data acquisition unit 10 described in FIG. 1.

The data storage unit 203 stores the biometric information measured by the sensor 201 and the time series data on the biometric information indicated by the digital signal obtained by the processing by the sensor data acquisition unit 202. The data storage unit 203 corresponds to the storage unit 12 (FIG. 1).

The transmission unit 204 transmits the biometric information stored in the data storage unit 203 to the relay terminal 300 through the communication network NW. The transmission unit 204 includes a communication circuit for performing wireless communication in compliance with wireless data communication standards such as LTE, 3G, a wireless local area network (LAN), or Bluetooth (trade name) for example. The transmission unit 204 corresponds to the transmission/reception unit 15 (FIG. 1).

### Functional Block of Relay Terminal

The relay terminal 300 includes a reception unit 301, a data storage unit 302, a data analysis unit 303, and a transmission unit 304. The relay terminal 300 analyzes the biometric information on the user received from the sensor terminal 200. Furthermore, the relay terminal 300 estimates the state of the user who performs the rehabilitation based on the biometric information on the user. Furthermore, the relay terminal 300 selects the corresponding mode of the spatiotemporally changing item, based on the state of the user estimated. The item of the selected mode is transmitted to the external terminal 400 as the rehabilitation support information.

The relay terminal 300 is implemented by a smart phone, a tablet, a laptop computer, a gateway, or the like.

The reception unit 301 receives the biometric information from the sensor terminal 200 through the communication network NW. The reception unit 301 corresponds to the transmission/reception unit 15 (FIG. 1).

The data storage unit 302 stores the biometric information on the user received by the reception unit 301 and history of the state of the user within the measurement period estimated by the data analysis unit 303. The data storage unit 302 corresponds to the storage unit 12 (FIG. 1).

The data analysis unit 303 analyzes the biometric information on the user received by the reception unit 301, estimates a state of the user involved in the rehabilitation, and selects the mode of the item such as a scene of the spatiotemporally changing movie in accordance with the progress of the rehabilitation based on the estimation result. The data analysis unit 303 corresponds to the data analysis unit 11 including the estimation unit no and the selection unit in described in FIGS. 1 and 2.

The transmission unit 304 transmits information indicating the mode of the spatiotemporally changing item selected by the data analysis unit 303 to the external terminal 400 through the communication network NW. The transmission unit 304 corresponds to the transmission/reception unit 15 (FIG. 1).

Functional Block of External Terminal

The external terminal 400 includes a reception unit 401, a data storage unit 402, a presentation processing unit 403, and a presentation unit 404. The external terminal 400 generates and presents the rehabilitation support information about the mode of the spatiotemporally changing item, based on the information received from the relay terminal 300 through the communication network NW.

Similar to the relay terminal 300, the external terminal 400 is implemented by a smart phone, a tablet, a laptop computer, a gateway, or the like. The external terminal 400 includes the display device 109 that generates and displays an image in accordance with the mode of the image of the received rehabilitation support information. Note that, in addition to the display device 109, the rehabilitation support information may be presented using a sound output device, a light source, or the like not illustrated in the drawings.

The reception unit 401 receives information indicating the mode of the spatiotemporally changing image, presented as the rehabilitation support information, from the relay terminal 300 through the communication network NW. The reception unit 401 corresponds to the transmission/reception unit 15 (FIG. 1).

The data storage unit 402 stores the mode of the spatiotemporally changing item. The data storage unit 402 corresponds to the storage unit 12 (FIG. 1).

The presentation processing unit 403 reads a mode of a spatiotemporally changing image, to be presented as the rehabilitation support information from the data storage unit 402, and outputs the mode. The presentation processing unit 403 can generate an image of the mode in accordance with the state of the user, such as the progress of the rehabilitation performed by the user, and control the display format of the rehabilitation support information. The presentation processing unit 403 may read a material such as an image, movie, sound, or the like set in advance, and may encode a result of editing including: combining the moving to be presented with sound or the like; setting playback speed; and processing using an effect filter. The presentation processing unit 403 corresponds to the presentation processing unit 13 illustrated in FIG. 1.

The presentation unit 404 outputs, as the rehabilitation support information, the selected mode of a spatiotemporally changing image, based on an instruction from the presentation processing unit 403. The presentation unit 404 may display the scene of the movie and the text information corresponding to the progress of the rehabilitation performed by the user on the display device 109, or output sound from a speaker (not illustrated) included in the external terminal 400. In addition, the presentation unit 404 can present the rehabilitation support information by a method perceptible by the user such as vibration, light, and stimulation. The presentation unit 404 corresponds to the presentation unit 14 described in FIG. 1.

As described above, the rehabilitation support system according to embodiments of the present invention has a configuration in which the functions illustrated in FIGS. 1 and 2 are distributed among the sensor terminal 200, the relay terminal 300, and the external terminal 400. The rehabilitation support system according to embodiments of the present invention is configured to execute, in a distributed manner, processing of: estimating the state of the user from the acquired biometric information on the user; selecting the mode such as the spatiotemporally changing image in accordance with the state of the user; and generating and presenting the image of the selected mode.

### Operating Sequence of Rehabilitation Support System

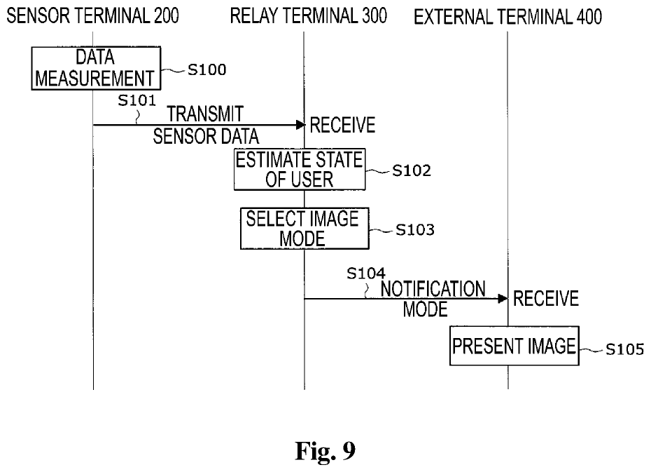

Next, operations of the rehabilitation support system having the above-described configuration will be described using the sequence diagram of FIG. 9.

As illustrated in FIG. 9, first, the sensor terminal 200 is attached to the user and measures biometric information such as three-axis acceleration for example (step S100). The sensor terminal 200 acquires a digital signal of the biometric information measured, and removes noise as necessary.

Next, the sensor terminal 200 transmits the biometric information to the relay terminal 300 through the communication network NW (step S101). Upon receiving the biometric information from the sensor terminal 200, the relay terminal 300 estimates the state of the user based on the biometric information (step S102). More specifically, the data analysis unit 303 of the relay terminal 300 calculates the state of the user from the biometric information, and records the biometric information together with the time information indicating the time when the biometric information, on which the state of the user is based, is measured.

Next, the data analysis unit 303 selects the mode of the item, such as a spatiotemporally changing image in accordance with the state of the user estimated in step S102 (step S103). Then, the relay terminal 300 transmits information indicating the selected mode of the image to the external terminal 400 through the communication network NW (step S104). Upon receiving the information indicating the mode of the image, the external terminal 400 performs the presentation processing for the image to be presented as the rehabilitation support information (step S105).

Now, an example of how the rehabilitation support information is presented on the external terminal 400 will be described with reference to FIG. 6 and FIG. 7. As illustrated in FIG. 6, the biometric information measured by the sensor terminal 200, for example, the measurement start time indicating when the acceleration of the user is measured, and the measurement time indicating when the latest data is measured are displayed on the external terminal 400. FIG. 6 and FIG. 7 illustrate an example in which a user is engaged in the rehabilitation of getting out of bed. The movie displayed with the scenes switching to show a story of a space craft launched from the earth to reach Neptune as the target destination, which represents the progress from the start of the rehabilitation performed by the user to the achievement the target level.

As illustrated in FIG. 6 and FIG. 7, on the display screen of the external terminal 400, an image of first arriving at the moon is displayed, when the out-of-bed time according to the history recorded in the out-of-bed state of the user is an hour. Then, when the out-of-bed state of the user falls within a range of 7 hours or longer and shorter than 14 hours, the displayed image is switched to an image of arrival at Mars. When the out-of-bed state of the user falls within a range of 14 hours or longer and shorter than 21 hours, the displayed image is switched to an image of arrival at Jupiter. When the out-of-bed state of the user reaches 21 hours, the displayed image is switched to an image of arrival at Saturn. When the out-of-bed state of the user reaches 28 hours, the displayed image is switched to an image of arrival at Uranus. When the out-of-bed state of the user reaches 35 hours, the displayed image is switched to an image of arrival at Neptune. In addition to the images indicating these points of arrival, the text information indicating the progress of the rehabilitation is presented.

In this manner, the scene of the movie presented switches to the next one in accordance with the progress of the rehabilitation, that is, each time the duration of the out-of-bed state of the user exceeds a duration set in advance.

As described above, the rehabilitation support system according to the first embodiment estimates the state of the user involved in the rehabilitation based on the biometric information on the user measured by the sensor 105, and selects and presents the mode of the spatiotemporally changing image in accordance with the state of the user thus estimated. Thus, the user can easily recognize the progress of the rehabilitation, to be more motivated toward the rehabilitation.

### Second Embodiment

Next, a second embodiment of the present invention will be described. In the following description, the same components as those in the first embodiment described above will be denoted by the same reference signs and description thereof will be omitted.

In the case described in the first embodiment, the data analysis unit 11 estimates, from the biometric information acquired by the sensor data acquisition unit 10, one state of a user involved in the rehabilitation, such as the out-of-bed state, for example. On the other hand, in the second embodiment, a data analysis unit 11 estimates a plurality of states of the user.

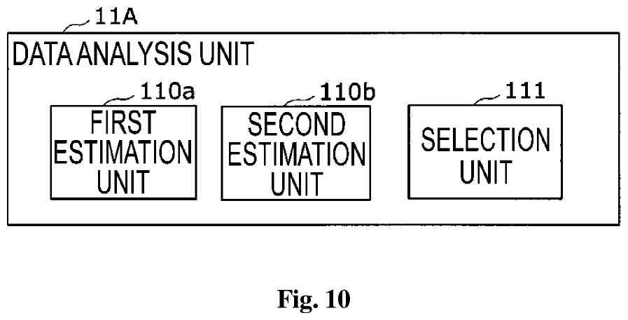

As illustrated in FIG. 10, the data analysis unit 11A of the rehabilitation support system according to the second embodiment is different from the configuration of the first embodiment, in that a first estimation unit 110a and a second estimation unit nob are provided. Hereinafter, components different from those of the first embodiment will be mainly described.

The first estimation unit 110a estimates the first state of the user involved in the rehabilitation, from the biometric information on the user acquired by the sensor data acquisition unit 10. For example, the first estimation unit 110a can estimate a parameter value indicating the first state of the user. The second estimation unit nob estimates a second state of the user, different from the first state, from the biometric information on the user acquired by the sensor data acquisition unit 10. For example, the second estimation unit 110a can estimate a parameter value indicating the second state of the user.

For example, the first estimation unit 110a estimates the out-of-bed state of the user from the acceleration of the user as the biometric information. On the other hand, the second estimation unit 110b estimates a body movement of the user, from the similarly acquired acceleration. The body movement of the user can, for example, be estimated to have occurred, when a moving standard deviation of the measurement value of the sensor 105 configured by an acceleration sensor within any time period, or a positive square root thereof is equal to or larger than a predetermined value. Specifically, for example, any time period may be two seconds, and the predetermined value may be 0.1 G.

In this manner, a plurality of states of the user different from each other are estimated based on biometric information, which is acceleration for example, acquired from one sensor 105.

The selection unit 111 selects the mode of the spatiotemporally changing item to be presented as the rehabilitation support information, based on the first state and the second state respectively estimated by the first estimation unit 110a and the second estimation unit nob. For example, the selection unit 111 can select the mode of an image in accordance with the parameter values of the first state and the second state.

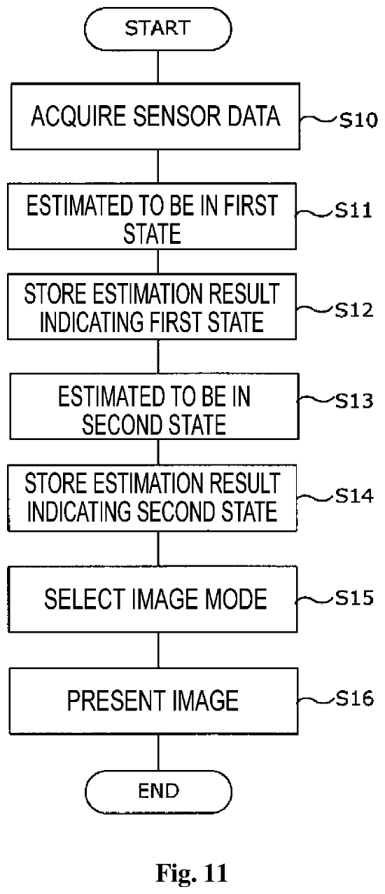

Next, the operation of the rehabilitation support system configured as described above will be described with reference to a flowchart of FIG. 11. First, the following processing is executed in a state where the sensor 105 is attached to the user.

The sensor data acquisition unit 10 acquires the biometric information on the user measured by the sensor 105 via the transmission/reception unit 15 (step S10). For example, acceleration data can be acquired as biometric information. The acquired biometric information is accumulated in the storage unit 12. Note that the sensor data acquisition unit 10 can execute processing of removing the noise in the biometric information acquired, and converting the analog signal of the biometric information into a digital signal.

Next, the first estimation unit 110a estimates the first state of the user based on the biometric information on the user acquired by the sensor data acquisition unit 10 (step Sn). For example, the first estimation unit 110a estimates that the user is in the out-of-bed state from the data indicating the acceleration of the user acquired by the sensor data acquisition unit 10. The result of the estimation by the first estimation unit 110a is stored in the storage unit 12 along with the time information (step S12).

Next, the second estimation unit nob estimates the second state of the user based on the biometric information on the user acquired by the sensor data acquisition unit 10 (step S13). For example, the second estimation unit nob estimates that the user has made a body movement, from the data indicating the acceleration of the user acquired by the sensor data acquisition unit 10. The result of the estimation by the second estimation unit nob is stored in the storage unit 12 along with the time information (step S14).

Thereafter, the selection unit 111 selects a mode of the spatiotemporally changing image to be presented as the rehabilitation support information, by using a history within any period from the start of measurement of sensor data based on which the state of the user is estimated by the first estimation unit 110a and the second estimation unit nob (step S15).

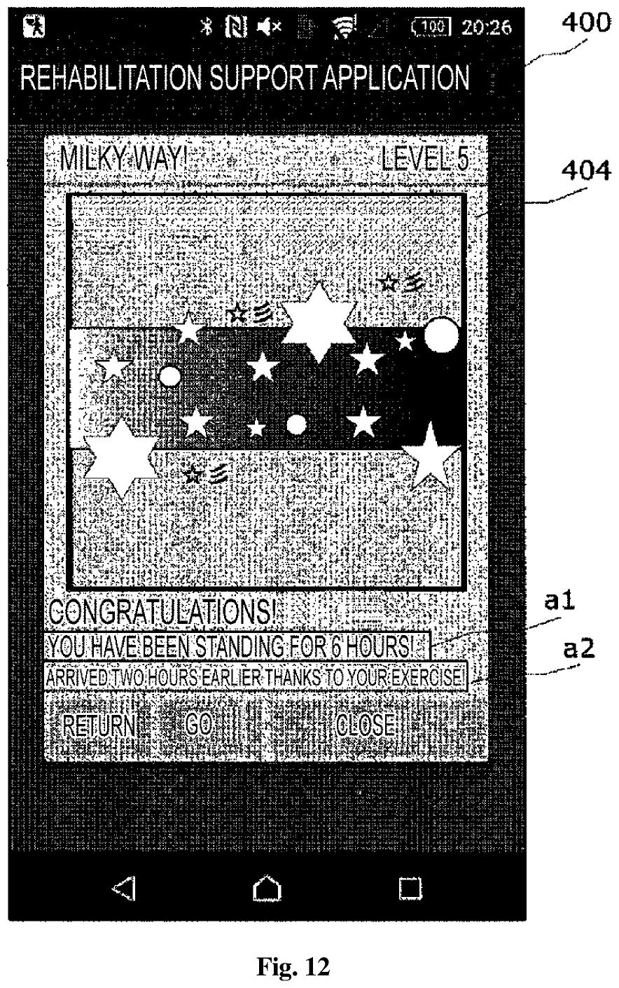

Specifically, as illustrated in the display example of the rehabilitation support information illustrated in FIG. 12, a mode of the image is selected from a history of the out-of-bed state estimated as the first state (item at in FIG. 12). Specifically, in the example illustrated in FIG. 12, image and text information representing “Milky way” and “Level 5” corresponding to the out-of-bed state which went on for six hours are selected. Furthermore, the value of the history of the body movement (item a2 in FIG. 12), corresponding to the second state, is doubled and the resultant value is added to the history of the first state.

Generally, the out-of-bed state can occur even when the user is not moving, but the body movement of the user can be regarded as having occurred in response to an active generation of acceleration. It can be generally regarded that when the body movement of the user occurs, an exercise load larger than that in the out-of-bed state is imposed. In view of this, the body movement corresponding to the second state is regarded as providing a higher rehabilitation effect in terms of rehabilitation of function than that in the out-of-bed state corresponding to the first state. Thus, the value is doubled as described above.

The state of the user such as the walking state indicated by the out-of-bed state and the body movement can have a parameter value set based on the exercise load and the effect of the rehabilitation on the rehabilitation of function.

Next, the presentation unit 14 causes the display device 109 to display the item of the selected mode as the rehabilitation support information, based on the mode of the spatiotemporally changing item selected by the selection unit 111 (step S16).

As described above, with the rehabilitation support system according to the second embodiment, a plurality of states of the user involved in the rehabilitation are estimated based on the same biometric information, and the mode of the spatiotemporally changing image in accordance with the estimation result is selected and presented. Thus, the user can be prompted to change his or her activities in a greater variety of ways.

Furthermore, the parameter value indicating each estimated state may be allocated with a set coefficient to weight selection of an image or the like presented as the rehabilitation support information, so that the user can be prompted to take more effective actions for the rehabilitation. As a result, the rehabilitation of the function of the user can be facilitated.

### Third Embodiment

Next, a third embodiment of the present invention will be described. In the following description, the same configurations as those in the first and the second embodiments described above will be denoted by the same reference signs and description thereof will be omitted.

In the cases described in the first and the second embodiments, the sensor data acquisition unit to acquires one biometric information measured by one sensor 105. On the other hand, in the third embodiment, a first acquisition unit bow and a second acquisition unit mob acquire the biometric information on the user respectively from sensors 105a and 105b different from each other.

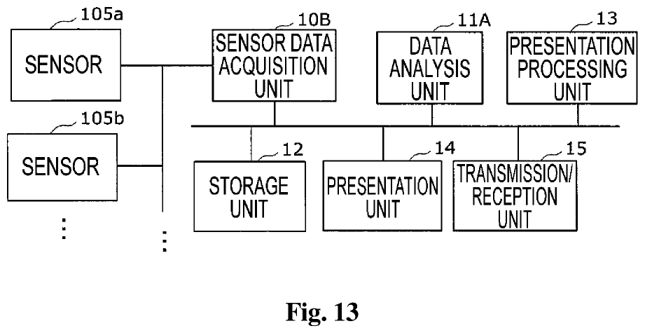

As illustrated in FIG. 13, the rehabilitation support system according to the third embodiment includes the plurality of sensors 105, a sensor data acquisition unit 10B, the data analysis unit 11A, the storage unit 12, the presentation processing unit 13, the presentation unit 14, and the transmission/reception unit 15.

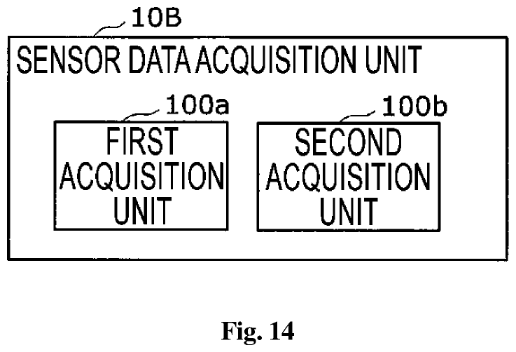

As illustrated in FIG. 14, the sensor data acquisition unit 10B includes the first acquisition unit bow and the second acquisition unit mob. The first acquisition unit 100a acquires first sensor data that is the biometric information on the user measured by the sensor 105a. For example, the first acquisition unit 100a acquires the acceleration of the user from the sensor 105a configured by the acceleration sensor.

The second acquisition unit mob acquires second sensor data that is the biometric information on the user measured by the sensor 105b. For example, the second acquisition unit mob acquires the heart rate of the user from the sensors 105b configured by the heart rate meter.

As in the configuration illustrated in FIG. 10, the data analysis unit 11A includes the first estimation unit 110a, the second estimation unit nob, and the selection unit 111.

The first estimation unit 110a estimates, based on the first sensor data acquired by the first acquisition unit bow, a first state indicating a state of the user involved in the rehabilitation. For example, the first estimation unit 110a can estimate the out-of-bed state and the like of the user from the acceleration of the user acquired as the first sensor data.

The second estimation unit 110b estimates, based on the second sensor data acquired by the second acquisition unit 100b, a second state indicating a state of the user. For example, the second estimation unit 110b can estimate an increase (change) in the heart rate of the user or the exercise load, based on the heart rate of the user acquired as the second sensor data.

Specifically, the second estimation unit 110b can compare the latest heart rate of the user acquired from the second acquisition unit 100b with the heart rate at rest stored in the storage unit 12 in advance. The second estimation unit 110b outputs an estimation result indicating that the load of the rehabilitation exercise is high, if the user's heart rate exceeds a predetermined value which is, for example, 40 bpm. Then, the second estimation unit 110b stores the event where the heart rate exceeds 40 bpm in the storage unit 12 together with the measurement time of the heart rate.

The selection unit 111 selects the mode of the spatiotemporally changing item, based on the first state and the second state of the user estimated by the first estimation unit 110a and the second estimation unit 110b. For example, when selecting a mode of an image based on the first state of the user estimated by the first estimation unit 110a, the selection unit 111 can select the mode of the spatiotemporally changing image while taking into consideration the information on the second state of the user estimated by the second estimation unit 110b.

For example, a case is assumed where when the first estimation unit 110a estimates the out-of-bed state and the magnitude of the body movement of the user, an estimation is made on the state of the user performing the rehabilitation under an exercise condition of holding a relatively heavy object for a period of time. In such a situation, active generation of acceleration is not expected to occur, and thus the state of the user is difficult to estimate using only the acceleration of the user measured by the acceleration sensor.

Thus, the second estimation unit 110b estimates the state of the user using biometric information on the user of a different type measured by the heart rate meter. Then, based on the state of the user more comprehensively estimated from the biometric information acquired from the plurality of different sensors 105a and 105b, the selection unit 111 can select the mode of the spatiotemporally changing item.

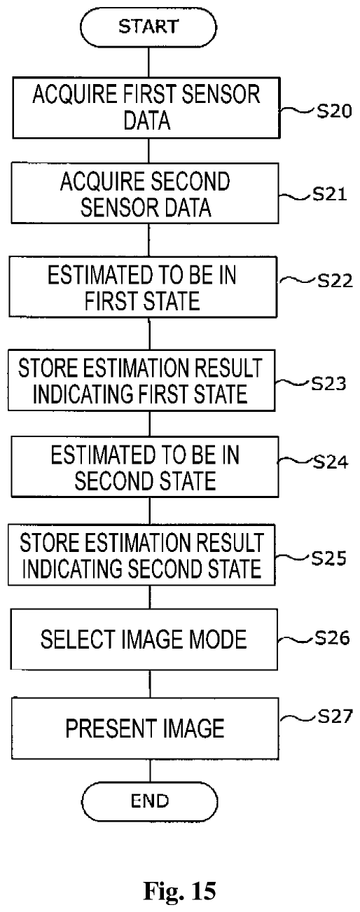

Next, the operation of the rehabilitation support system according to the present embodiment will be described with reference to a flowchart of FIG. 15. First, the following processing is executed in a state where the sensors 105a and 105b are attached to the user.

The first acquisition unit tow acquires the first sensor data which is the biometric information on the user measured by the sensor 105a via the transmission/reception unit 15 (step S20). For example, acceleration data can be acquired as the first sensor data. The acquired biometric information is accumulated in the storage unit 12. Note that the first acquisition unit tow can execute processing of remove the noise in the first sensor data acquired, and converting the analog signal of the biometric information into a digital signal.

Next, the second acquisition unit mob acquires the second sensor data which is the biometric information on the user measured by the sensor 105b via the transmission/reception unit 15 (step S21). For example, heart rate can be acquired as the second sensor data. The acquired second sensor data is accumulated in the storage unit 12.

Next, the first estimation unit 110a estimates the first state of the user based on the first sensor data on the user acquired by the first acquisition unit tow (step S22). For example, the first estimation unit 110a estimates that the user is in the out-of-bed state from the acceleration of the user. The result of the estimation by the first estimation unit 110a is stored in the storage unit 12 along with the time information (step S23).

Next, the second estimation unit nob estimates the second state of the user based on the biometric information on the user acquired by the sensor data acquisition unit to (step S24). For example, the second estimation unit nob estimates a change in the heart rate or the exercise load of the user, based on the change in heart rate of the user acquired by the second acquisition unit 100b. The result of the estimation by the second estimation unit 110b is stored in the storage unit 12 along with the time information (step S25).

Thereafter, the selection unit 111 selects a mode of the image to be presented as the rehabilitation support information, by using a history within any period from the start of measurement of the first sensor data and the second sensor data based on which the first state and the second state of the user are estimated by the first estimation unit 110a and the second estimation unit nob (step S26).

Note that the selection unit 111 may select different images respectively corresponding to the first state and the second state. For example, for the first state, a mode of an image representing a passing point of a space craft traveling in space is selected. For the second state, another image element may be selected which is a mode of an image representing indicating the number of and twinkling of stars for example. Alternatively, when an image of a plurality of animals and plants are presented, the selection unit 111 can select the modes of the animals and plants respectively corresponding to the first state and the second state.

Next, the presentation unit 14 causes the display device 109 to display the image of the selected mode as the rehabilitation support information, based on the mode of the spatiotemporally changing image selected by the selection unit 111 (step S27).

Although the case is described where the second acquisition unit mob acquires the heart rate of the user from the sensor 105b configured by the heart rate meter, the biometric information acquired by the second acquisition unit mob may be blood pressure, respiratory rate, sweating, or the like of the user instead of the heart rate.

Although the sensors 105a and 105b are different types of sensors that are described as an example, these sensors may be sensors of the same type. For example, an acceleration sensor attached to the trunk of the user cannot measure the movement of the limbs of the body, such as arms and legs of the user. Thus, acceleration sensors may be respectively provided to the trunk and the limbs such as the arms and the legs, to acquire acceleration of a plurality of parts of the body.

In the case described in the above embodiment, the first acquisition unit tow and the second acquisition unit mob acquire the first sensor data and the second sensor data from the two sensors 105a and 105b respectively. However, the number of sensors and the number of acquisition units are not limited to two, and may be three or more.

As described above, with the rehabilitation support system according to the third embodiment, a plurality of states of the user involved in the rehabilitation are estimated based on a plurality of types of biometric information obtained from the user performing the rehabilitation, and the mode of the spatiotemporally changing image in accordance with the estimation result is selected and presented. Thus, the rehabilitation support system according to the third embodiment can more comprehensively estimate the state of the user, and present the rehabilitation support information more accurately reflecting the activity and effort of the user working on the rehabilitation. As a result, the rehabilitation support system according to the third embodiment can support the rehabilitation to be more satisfactory to the user.

### Fourth Embodiment

Next, a fourth embodiment of the present invention will be described. In the following description, the same configurations as those in the first to third embodiments described above will be denoted by the same reference signs and description thereof will be omitted.

In the cases described in the first to the third embodiments, the data analysis unit 11 estimates the state of the user involved in the rehabilitation. On the other hand, a data analysis unit 11C according to the fourth embodiment detects whether the user is performing the rehabilitation. Then, when it is detected that the user is not performing the rehabilitation, a mode of a spatiotemporally changing item is selected based on the length of a period during which the user is not performing the rehabilitation.

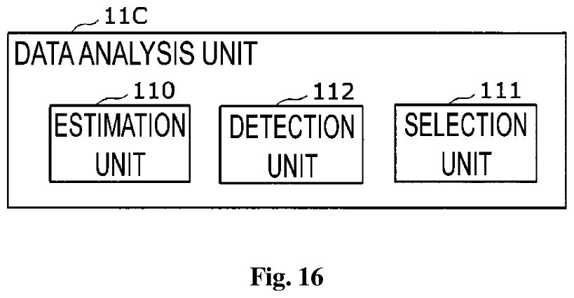

As illustrated in FIG. 16, the data analysis unit 11C includes the estimation unit 110, the selection unit tit, and a detection unit 112. The other configuration of the rehabilitation support system according to the present embodiment is the same as that in the first embodiment.

The estimation unit 110 estimates the state of the user involved in the rehabilitation, from the biometric information on the user acquired by the sensor data acquisition unit to. For example, when the sensor 105 is configured by an acceleration sensor, the out-of-bed state is estimated from the acceleration of the user. The state of the user estimated is stored in the storage unit 12 together with the time information indicating when the biometric information has been measured by the sensor 105.

The detection unit 112 detects whether the user is performing rehabilitation. More specifically, when the state of the user involved in the rehabilitation is estimated by the estimation unit 110, the detection unit 112 detects that the rehabilitation is being performed. On the other hand, when the state of the user involved in the rehabilitation is not estimated by the estimation unit 110, the detection unit 112 detects that the rehabilitation is not being performed. For example, when the estimation unit no estimates the out-of-bed state of the user, the user in other states, such as the lying state for example, is detected to be not performing the rehabilitation.

The selection unit 111 selects the mode of a spatiotemporally changing image, in accordance with the state of the user estimated by the estimation unit 110. When the detection unit 112 detects that the user is not performing the rehabilitation, the selection unit 111 selects the mode of the image based on the length of the period the user is not performing the rehabilitation.

For example, the estimation unit no estimates the out-of-bed state of the user. In this case, the detection unit 112 detects a period in which the user is not in the out-of-bed state and thus is in the lying state. When the period in which the user is not in the out-of-bed state detected by the detection unit 112 is equal to or a longer than a predetermined period, which is two hours for example, the selection unit 111 can select a mode of an image indicating an alert as the rehabilitation support information.

Specifically, when the detected period during which the user is not in the out-of-bed state is two hours in a case where the movie of a space craft traveling is displayed as the rehabilitation support information with an image switched based on the history of the out-of-bed state of the user, an image of an alien attempting to attack the space craft or an image of the space craft deviating off course may be presented for example. Alternatively, a penalty may be calculated, and a mode of an image requiring the user to stay in the out-of-bed state for a longer period of time can be selected, when switching to an image of the space craft arriving at the next planet.

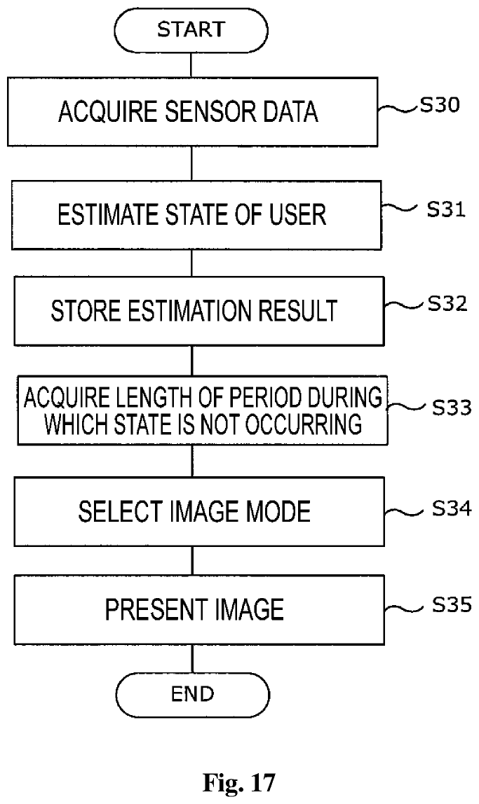

Next, the operation of the rehabilitation support system according to the present embodiment will be described with reference to a flowchart of FIG. 17. First, the following processing is executed in a state where the sensor 105 is attached to the user.

The sensor data acquisition unit 10 acquires the biometric information on the user measured by the sensor 105 via the transmission/reception unit 15 (step S3o). For example, acceleration data can be acquired as biometric information. The acquired biometric information is accumulated in the storage unit 12. Note that the sensor data acquisition unit 10 can execute processing of remove the noise in the biometric information acquired, and converting the analog signal of the biometric information into a digital signal.

Next, the estimation unit no estimates the state of the user based on the biometric information acquired by the sensor data acquisition unit 10 (step S31). For example, the estimation unit no estimates that the user is in the out-of-bed state from the acceleration of the user. The result of the estimation by the estimation unit 110 is stored in the storage unit 12 along with the time information (step S32).

Next, the detection unit 112 detects that the user is not performing rehabilitation (step S33). The detection unit 112 can detect as a period during which the user is not performing the rehabilitation, a period during which a predetermined state of the user is not estimated by the estimation unit no. For example, the detection unit 112 can calculate the period during which the user is not in the out-of-bed state from the history of the state of the user stored in the storage unit 12.

Thereafter, the selection unit 111 selects a mode of a spatiotemporally changing image, based on the history of the state of the user estimated by the estimation unit no and information on the period during which the user is not in the state (step S34). Specifically, when the period during which the state of the user is not estimated reaches or exceeds a predetermined period, the selection unit 111 selects an image indicating an alert set in advance.

When the estimation unit no estimates the state of the user involved in the rehabilitation, the detection unit 112 detects that the user is performing the rehabilitation. Thus, as in the first to the third embodiments, the selection unit 111 selects the mode of an image presented as the rehabilitation support information, based on the history of the state of the user involved in the rehabilitation.

Next, the presentation unit 14 causes the display device 109 to display the image representing the rehabilitation support information, based on the mode of the spatiotemporally changing image selected by the selection unit 111 (step S35).

As described above, with the rehabilitation support system according to the fourth embodiment, a spatiotemporally changing image is selected and presented based on a period in which the user is not in a predetermined state involved in the rehabilitation. Thus, with the rehabilitation support system according to the fourth embodiment, a preventive rehabilitation support can be implemented to prevent the state which is regarded to be unfavorable in terms of effective rehabilitation from continuing for an excessively long period of time. As a result, the rehabilitation support system according to the fourth embodiment can further motivate the user to work on his or her rehabilitation.

### Fifth Embodiment

Next, a fifth embodiment of the present invention will be described. In the following description, the same configurations as those in the first to fourth embodiments described above will be denoted by the same reference signs and description thereof will be omitted.

In the cases described in the first to the fourth embodiments, the data analysis unit 11 estimates the state of the user involved in the rehabilitation. On the other hand, in the fifth embodiment, the data analysis unit 11 selects a mode of a spatiotemporally changing item, in response to the state of the user involved in the rehabilitation continuing for a predetermined period.

The configuration of the rehabilitation support system according to the present embodiment is the same as that in the first embodiment.

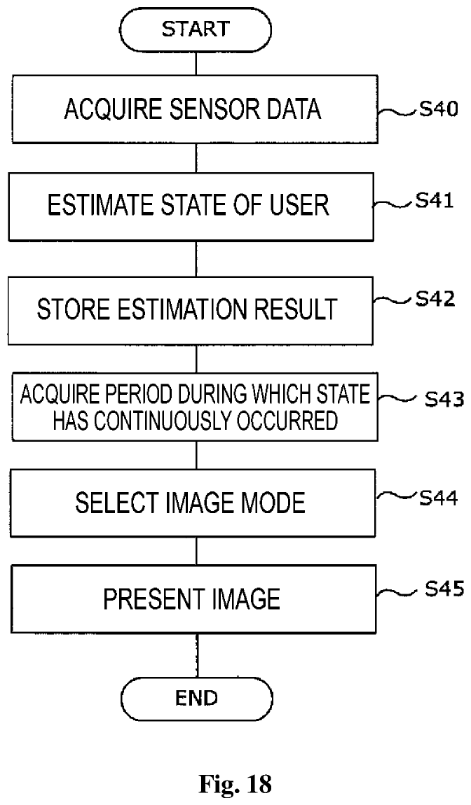

FIG. 18 is a flowchart illustrating an operation of the rehabilitation support system according to the present embodiment. First, the following processing is executed in a state where the sensor 105 is attached to the user.

The sensor data acquisition unit 10 acquires the biometric information on the user measured by the sensor 105 via the transmission/reception unit 15 (step S40). For example, acceleration data can be acquired as biometric information. The acquired biometric information is accumulated in the storage unit 12. Note that the sensor data acquisition unit 10 can execute processing of remove the noise in the biometric information acquired, and converting the analog signal of the biometric information into a digital signal.

Next, the estimation unit no estimates the state of the user based on the biometric information acquired by the sensor data acquisition unit 10 (step S41). For example, the estimation unit no estimates that the user is in the out-of-bed state from the acceleration of the user. The result of the estimation by the estimation unit 110 is stored in the storage unit 12 along with the time information (step S42).

Next, the selection unit 111 reads a period during which the state of the user has continued, based on the history of the state of the user stored in the storage unit 12 (step S43). For example, the selection unit 111 can acquire the length of the period during which the user continues to be in the out-of-bed state, based on the latest history of the state of the user stored in the storage unit 12.

Then, the selection unit 111 selects the mode of the spatiotemporally changing image, by using the length of the period during which the state of the user continues acquired in step S43 (step S44).

Next, the presentation unit 14 causes the display device 109 to display the image of the selected mode as the rehabilitation support information, based on the information indicating the image of the mode selected by the selection unit 111 (step S45).

Note that the selection unit 111 may select the mode of the spatiotemporally changing image, by acquiring the latest period during which the state of the user has been continuously absent, from the history of the period during which the user is not performing the rehabilitation, as described in the fourth embodiment.

For example, the expected effect of the rehabilitation is different between a case where a cumulative value of the state of the user intermittently occurring exceeds a predetermined amount and a case where a cumulative value of the state of the user continuously occurring exceeds a predetermined amount. In the former case, an average load of exercise involved in the rehabilitation, in the lifestyle of the user, is expected to improve. On the other hand, the latter case is likely to take place to increase a load of exercise involved in the rehabilitation performed by the user over a shorter period of time.

With the period during which the state of the user, involved in the rehabilitation, continues thus used as a condition for selecting a mode of a spatiotemporally changing image as in the rehabilitation support system according to the fifth embodiment, the load in a rehabilitation menu performed by the user in a shorter period of time can be expected to be improved.

Note that the described embodiments can be implemented in combination. The rehabilitation support systems according to the second to the fifth embodiments may be achieved by the sensor terminal 200, the relay terminal 300, and the external terminal 400 illustrated in FIG. 5, FIG. 8, and FIG. 9 as in the first embodiment.

Furthermore, in the above description, the data analysis unit 11 is included in the relay terminal 300 in the rehabilitation support system achieved by the sensor terminal 200, the relay terminal 300, and the external terminal 400. Alternatively, the data analysis unit 11 may be included in the sensor terminal 200 or the external terminal 400.

The functions (the estimation unit no and the selection unit 111) of the data analysis unit 11 may be distributed among the sensor terminal 200, the relay terminal 300, and the external terminal 400 to be implemented.

Although the embodiment of the rehabilitation support system, the rehabilitation support method, and the rehabilitation support program of embodiments of the present invention have been described above, the present invention is not limited to the described embodiments, and various types of modification that can be conceived by a person skilled in the art can be made within the scope of the invention.

### REFERENCE SIGNS LIST

10, 202 Sensor data acquisition unit
11, 303 Data analysis unit
12 Storage unit
13, 403 Presentation processing unit
14, 404 Presentation unit
15 Transmission/reception unit
110 Estimation unit
111 Selection unit
101 Bus
102 Processor
103 Main storage device
104 Communication interface
105, 201 Sensor
106 Auxiliary storage device
107 Timepiece
108 Input/output device
109 Display device
200 Sensor terminal
300 Relay terminal
400 External terminal
203, 302, 402 Data storage unit
204, 304 Transmission unit
301, 401 Reception unit

1.-8. (canceled)

9. A rehabilitation support system comprising:
a sensor data acquirer configured to acquire biometric information on a user, the biometric information measured by a plurality of sensors;
an estimator configured to estimate a state of the user based on the biometric information;
a storage device configured to store modes of a spatiotemporally changing item;
a selector configured to select a first mode of the spatiotemporally changing item, in accordance with the state of the user estimated by the estimator, from the modes of the spatiotemporally changing item stored in the storage device; and
a presenter configured to present the first mode of the spatiotemporally changing item selected by the selector.

10. The rehabilitation support system of claim 9, wherein the presenter comprises a display device configured to display an image representing the first mode of the spatiotemporally changing item in accordance with the state of the user.

11. The rehabilitation support system of claim 9, wherein:
the sensor data acquirer is configured to acquire the biometric information on the user from each of the plurality of sensors;
the estimator is configured to estimate values of a plurality of parameters indicating the state of the user, based on the biometric information acquired from each of the plurality of sensors; and
the selector is configured to select the first mode of the spatiotemporally changing item in accordance with the estimated values of the plurality of parameters.

12. The rehabilitation support system of claim 11, wherein the selector is configured to select the first mode of the spatiotemporally changing item by weighting the estimated values of the plurality of parameters.

13. The rehabilitation support system of claim 11, wherein the plurality of parameters comprise an activity state, a magnitude of body movement, and a change in heart rate of the user.

14. The rehabilitation support system of claim 9 further comprising:
a detector configured to detect whether the user is performing a rehabilitation,
wherein the selector is configured to select, when the detector detects that the user is performing the rehabilitation, the first mode of the spatiotemporally changing item in accordance with the state of the user estimated by the estimator, and to select, when the detector detects that the user is not performing the rehabilitation, the first mode of the spatiotemporally changing item in accordance with a length of a period during which the user is not performing the rehabilitation.

15. A rehabilitation support method comprising:
acquiring biometric information on a user, the biometric information measured by a plurality of sensors;
estimating a state of the user based on the biometric information;
selecting a first mode of a spatiotemporally changing item, in accordance with the state of the user, from modes of the spatiotemporally changing item stored in a storage device; and
presenting the first mode of the spatiotemporally changing item.

16. The rehabilitation support method of claim 15, wherein presenting the first mode of the spatiotemporally changing item comprises displaying an image representing the first mode of the spatiotemporally changing item in accordance with the state of the user.

17. The rehabilitation support method of claim 15, wherein the biometric information on the user is acquired from each of the plurality of sensors, the rehabilitation support method further comprising:
estimating values of a plurality of parameters indicating the state of the user, based on the biometric information acquired from each of the plurality of sensors; and
selecting the first mode of the spatiotemporally changing item in accordance with the estimated values of the plurality of parameters.

18. The rehabilitation support method of claim 17, wherein selecting the first mode of the spatiotemporally changing item comprises weighting the estimated values of the plurality of parameters.

19. The rehabilitation support method of claim 17, wherein the plurality of parameters comprise an activity state, a magnitude of body movement, and a change in heart rate of the user.

20. The rehabilitation support method of claim 15 further comprising:
detecting whether the user is performing a rehabilitation;
selecting, in response to detecting that the user is performing the rehabilitation, the first mode of the spatiotemporally changing item in accordance with the state of the user; and
selecting, in response to detecting that the user is not performing the rehabilitation, the first mode of the spatiotemporally changing item in accordance with a length of a period during which the user is not performing the rehabilitation.

21. A non-transitory computer readable storage medium storing a rehabilitation support program for execution by a processor, the rehabilitation support program comprising instructions for:
acquiring biometric information on a user, the biometric information measured by a plurality of sensors;
estimating a state of the user based on the biometric information;
selecting a first mode of a spatiotemporally changing item, in accordance with the state of the user, from modes of the spatiotemporally changing item stored in a storage device; and
presenting the first mode of the spatiotemporally changing item.

22. The non-transitory computer readable storage medium of claim 21, wherein the instruction for presenting the first mode of the spatiotemporally changing item comprises instructions for displaying an image representing the first mode of the spatiotemporally changing item in accordance with the state of the user.

23. The non-transitory computer readable storage medium of claim 21, wherein the biometric information on the user is acquired from each of the plurality of sensors, and the rehabilitation support program further comprises instructions for:
estimating values of a plurality of parameters indicating the state of the user, based on the biometric information acquired from each of the plurality of sensors; and
selecting the first mode of the spatiotemporally changing item in accordance with the estimated values of the plurality of parameters.

24. The non-transitory computer readable storage medium of claim 23, wherein the instruction for selecting the first mode of the spatiotemporally changing item comprises instructions for weighting the estimated values of the plurality of parameters.

25. The non-transitory computer readable storage medium of claim 23, wherein the plurality of parameters comprise an activity state, a magnitude of body movement, and a change in heart rate of the user.

26. The non-transitory computer readable storage medium of claim 21, wherein the rehabilitation support program further comprises instructions for:
detecting whether the user is performing a rehabilitation;
selecting, in response to detecting that the user is performing the rehabilitation, the first mode of the spatiotemporally changing item in accordance with the state of the user; and
selecting, in response to detecting that the user is not performing the rehabilitation, the first mode of the spatiotemporally changing item in accordance with a length of a period during which the user is not performing the rehabilitation.
